(19) United States
(12) Patent Application Publication
(10) Pub. No.: US 2015/0094626 A1
(43) Pub. Date: Apr. 2, 2015

(54) ELECTRODE PACKAGE WITH FOLD OUT LINER

(71) Applicant: ZOLL MEDICAL CORPORATION, Chelmsford, MA (US)

(72) Inventors: Deborah T. Jones, Dartmouth, MA (US); Christopher Desmarais, Acushnet, MA (US); David N. Craige, III, Attleboro, MA (US)

(21) Appl. No.: 14/494,237

(22) Filed: Sep. 23, 2014

Related U.S. Application Data
(60) Provisional application No. 61/883,273, filed on Sep. 27, 2013.

(51) Int. Cl. A61N 1/04; A61H 31/00

(52) U.S. Cl. CPC A61N 1/046; A61H 31/00
USPC 601/41; 607/142

(57) ABSTRACT

Methods and systems of applying treatment to a subject experiencing cardiac distress. In one example, a therapy electrode package includes a first therapy electrode disposed within the package, a second therapy electrode disposed within the package, and a CPR assistance device disposed within the package. The first therapy electrode, the second therapy electrode, and the CPR assistance device are arranged within the therapy electrode package such that when the electrode package is in an open configuration, the relative positions of the first therapy electrode, the second therapy electrode, and the CPR assistance device approximate the relative positions at to which the first therapy electrode, the second therapy electrode, and the CPR assistance device should be placed on a subject for treatment of the subject.

### RELATED APPLICATIONS

This application claims priority under 35 U.S.C. §119(e) to U.S. Provisional Application Ser. No. 61/883,273, titled “ELECTODE PACKAGE WITH FOLD OUT LINER,” filed on Sep. 27, 2013, which is herein incorporated by reference in its entirety.

### BACKGROUND

1. Technical Field

Aspects and embodiments of the present invention relate to systems and methods for the treatment of individuals experiencing cardiac distress, and in particular to therapy electrodes and packaging of therapy electrodes.

2. Discussion of Related Art

Treatment of a subject experiencing cardiac distress may generally include clearing the subject's airway, assisting the subject's breathing, chest compressions, and defibrillation.

Defibrillation can be performed with the use of an automatic external defibrillator (AED). Most automatic external defibrillators are actually semi-automatic external defibrillators (SAED), which require a clinician to press a start button, after which the defibrillator analyzes the subject's condition and provides a shock to the subject if the electrical rhythm is shockable and waits for user intervention before any subsequent shock. Fully automatic external defibrillators, on the other hand, do not wait for user intervention before applying subsequent shocks. As the term is used herein, automatic external defibrillators (AED) include semi-automatic external defibrillators (SAED).

Both types of defibrillators typically provide an oral stand clear warning before the application of each shock, and then the clinician or first responder is expected to stand clear of the subject and may be required to press a button indicating that the clinician is standing clear of the subject. The controls for automatic external defibrillators are typically located on a control panel of the defibrillator.

AEDs are typically used by trained providers such as physicians, nurses, fire department personnel, and police officers. There might be one or two people at a given facility that has an AED who have been trained to provide emergency aid, such as defibrillation and/or resuscitation before an ambulance service arrives. The availability of on-site AEDs along with rescuers trained to operate them is important because if the subject experiences a delay of more than about 90 seconds before receiving a defibrillation shock the subject's chance of survival can drop dramatically. It is well documented that for every minute defibrillation is delayed after onset of a shockable cardiac rhythm in a subject, the chance of survival of the subject falls 7 to 10 percent. Many large cities and rural areas have low survival rates for defibrillation because the ambulance response time is slow. Many suburbs have higher survival rates because of the faster ambulance response time due to lack of traffic and availability of hospitals and advanced life support.

Trained lay providers are a new group of AED operators, but they rarely have opportunities to defibrillate. For example, spouses of heart attack victims may become lay providers, but these lay providers can be easily intimidated by an AED during a medical emergency. Consequently, such lay providers can be reluctant to purchase AEDs, or might tend to wait for an ambulance to arrive rather than use an available AED, out of concern that the lay provider might do something wrong.

There is a growing trend toward the replacement of multiple use defibrillator paddles with single-use disposable therapeutic electrodes for defibrillation, external transthoracic pacing, or a combination of both. This trend is driven by numerous factors including, but not limited to: (1) convenience related to not having to apply a conductive media (e.g., gel), (2) speed of care when switching from delivering a defibrillation shock to a pacing current, (3) caregiver safety in that contact with the subject can be avoided as the therapy can be delivered remotely from the host device, and (4) increased use of defibrillators incorporating algorithms that analyze the presented ECG rhythm for appropriateness of therapeutic (shock) delivery.

### SUMMARY

In accordance with a first aspect of the present disclosure, there is provided a therapy electrode package. The therapy electrode package comprises a first therapy electrode disposed within the package, a second therapy electrode disposed within the package, and a CPR assistance device disposed within the package. The first therapy electrode, the second therapy electrode, and the CPR assistance device are arranged within the therapy electrode package such that when the electrode package is in an open configuration, the relative positions of the first therapy electrode, the second therapy electrode, and the CPR assistance device approximate the relative positions at which the first therapy electrode, the second therapy electrode, and the CPR assistance device should be placed on a subject for treatment of the subject to facilitate or ensure optimal treatment of the subject.

In some embodiments, the therapy electrode package further comprises a liner which defines a pocket in which the first therapy electrode, the second therapy electrode, and the CPR assistance device are retained when the package is in a closed configuration. The liner may include a mechanism for maintaining the liner in a folded configuration absent application of a force to open the package. The mechanism may provide the pocket with an air-tight seal when the package is in a closed configuration.

In some embodiments, the CPR assistance device extends at least partially outside a perimeter defined by the liner while the package is in an open configuration. The liner may include a first side having a first internal surface and a second side including a second internal surface. The CPR assistance device may be adhered to a material having a first end coupled to the first internal surface and a second end coupled to the second internal surface. The material may displace the CPR assistance device from a position within the pocket to the position at least partially outside the perimeter defined by the liner upon opening the package.

In some embodiments, one of the first electrode and the second electrode are retained in a pocket defined within the package. In other embodiments, one of the first electrode and the second electrode are removably adhered to a coating disposed on an inner surface of the liner.

In some embodiments, the therapy electrode package may be disposed within a pocket defined in a defibrillator.

In accordance with another aspect, there is provided a method of providing treatment to a subject experiencing cardiac distress. The method comprises obtaining a defibrillator and an electrode package, opening the electrode package to access electrodes and a CPR assistance device within the electrode package, determining proper placement of the electrodes and CPR assistance device on the subject from relative positions of the electrodes and CPR assistance device within the opened package, placing the electrodes and CPR assistance device on the subject, and performing treatment on the subject, the treatment including at least one of application of CPR and delivery of a defibrillation pulse to the subject. Opening the electrode package may include disconnecting portions of a sealing mechanism configured to maintain the therapy electrode package in a closed state until the therapy electrode package is opened by a user.

The method may further comprise electrically coupling the electrodes to the defibrillator and/or may further comprise removing the electrode package from one of a pocket attached to the defibrillator and a slot defined in the defibrillator.

In accordance with another aspect, there is provided a therapy electrode package. The therapy electrode package comprises a first therapy electrode, a second therapy electrode, and a CPR assistance device. The therapy electrode package has a closed state in which the first therapy electrode, the second therapy electrode, and the CPR assistance device are disposed within the package and an open state. The CPR assistance device is moved by a portion of the package to a position extending at least partially beyond a periphery of the package upon a transition of the package from the closed state to the open state.

In some embodiments, the first therapy electrode, the second therapy electrode, and the CPR assistance device are arranged within the therapy electrode package such that when the electrode package is in the open state, the relative positions of the first therapy electrode, the second therapy electrode, and the CPR assistance device approximate the relative positions at which the first therapy electrode, the second therapy electrode, and the CPR assistance device should be placed on a subject for treatment of the subject to facilitate or ensure optimal treatment of the subject. At least one of the first therapy electrode and the second therapy electrode may be releasably adhesively retained on a coating of a liner of the therapy electrode package. The portion of the package may comprise a piece of material releasably coupled to the CPR assistance device and having opposite ends coupled to opposite internal sides of the package.

The therapy electrode package may further include means for maintaining the therapy electrode package in a closed state until the therapy electrode package is opened by a user. The means for maintaining the therapy electrode package in the closed state may form an air-tight seal about a pocket defined in the package in the closed state, the pocket housing at least one of the first therapy electrode, the second therapy electrode, and the CPR assistance device.

### BRIEF DESCRIPTION OF THE DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a drawing of an embodiment of a defibrillator;

FIG. 2 is a plan view of an embodiment of an electrode package;

FIG. 3A illustrates a step in a sequence of opening the electrode package of FIG. 2;

FIG. 3B illustrates a step in a sequence of opening the electrode package of FIG. 2;

FIG. 3C illustrates a step in a sequence of opening the electrode package of FIG. 2;

FIG. 3D illustrates a step in a sequence of opening the electrode package of FIG. 2;

FIG. 4A illustrates a label for an electrode included in the electrode package of FIG. 2;

FIG. 4B illustrates a label for another electrode included in the electrode package of FIG. 2;

FIG. 4C illustrates a label for a CPR assistance device included in the electrode package of FIG. 2;

FIG. 5 illustrates an embodiment of an electrode package in an open position;

FIG. 6 illustrates another embodiment of an electrode package in an open position; and

FIG. 7 is a flowchart of a method disclosed herein.

### DETAILED DESCRIPTION

Aspects and embodiments of the present invention are not limited to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” “having,” “containing,” “involving,” and variations thereof herein is meant to encompass the items listed thereafter and equivalents thereof.

Aspects and embodiments of the present invention include systems and methods which may facilitate the rapid delivery of treatment to an individual experiencing cardiac distress, thus increasing the chance of survival of the individual. It has been recognized that the removal of therapy electrodes and/or CPR assistance devices from some prior known packages and the placement of the therapy electrodes and/or CPR assistance devices on appropriate areas of the body of a subject may be a source of delay in the provision of treatment to a subject experiencing cardiac distress. This delay may be especially pronounced if the treatment is being provided to the subject by a layperson not familiar with the proper placement of therapy electrodes and/or CPR assistance devices on a subject.

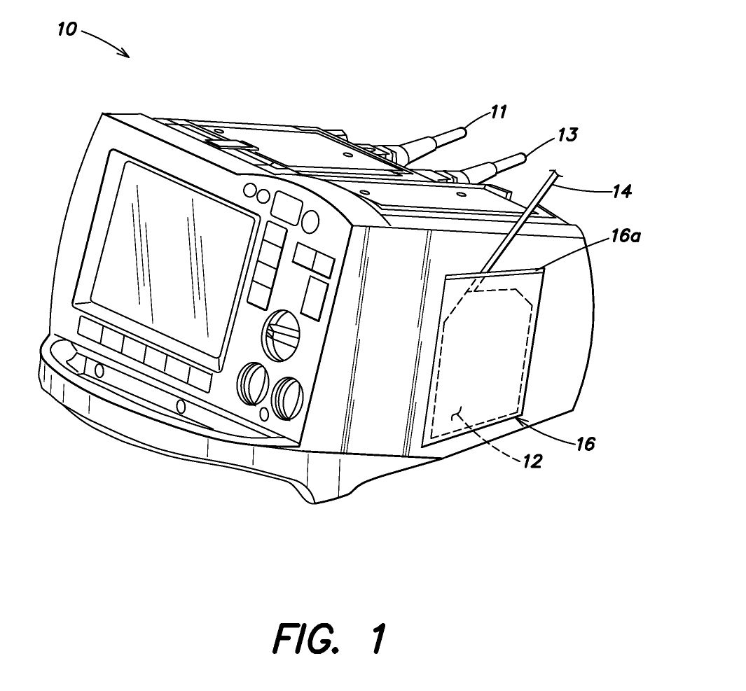

FIG. 1 shows an external defibrillator 10 similar to an R Series external defibrillator manufactured by ZOLL Medical Corporation. User interface elements (graphical display, speaker, microphone, input buttons and dials) are provided on the front face of the defibrillator. Attached to the right side of the defibrillator is a holder or pocket 16 for retaining an electrode package 12. The electrode package 12 is removable from the holder or pocket 16 through a slot 16a in the holder or pocket 16. In some embodiments, electrodes within the electrode package 12 are normally electrically connected to the defibrillator by a multi-conductor cable 14 even when the defibrillator is not in use. The multi-conductor cable 14 emerging from the electrode package may be divided into two cables 11, 13 which attach to the back of the defibrillator.

The defibrillator 10 is illustrated as an example of a defibrillator with which embodiments of an electrode package as disclosed herein may be utilized. Other defibrillators, for example, defibrillators similar to the AED Plus® Automated External Defibrillator or AED Pro® Automated External Defibrillator manufactured by ZOLL Medical Corporation may be provided with holders or pockets 16 or slots for retaining electrode packages as disclosed herein. Alternatively, electrode packages as disclosed herein may be provided separately from defibrillators and may include connectors for quick connection to an available defibrillator when needed.

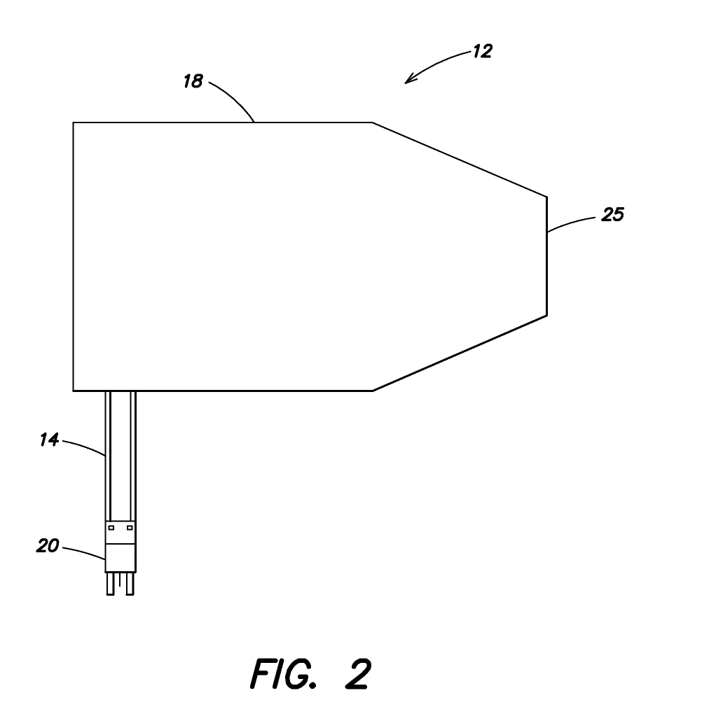

Embodiments of an electrode package 12 may be provided in a closed folded configuration, for example, as illustrated in FIG. 2. A package liner 18 may be folded over itself to form a pocket in which electrodes and/or a CPR assistance deice may be retained. A multi-conductor cable 14 terminating in an electrical connector 20 may extend outward from the pocket defined by the folded liner 18, or in some embodiments, may be retained internal to the pocket defined by the folded liner 18.

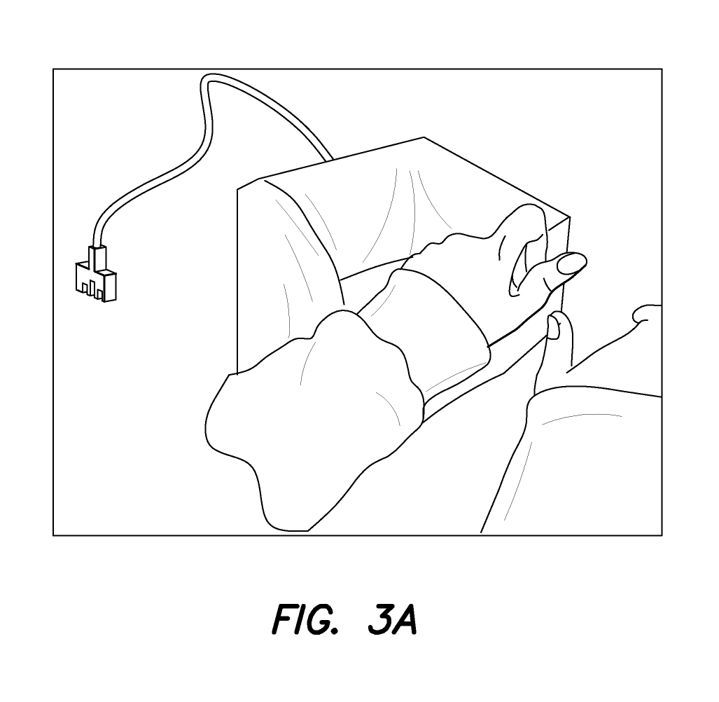

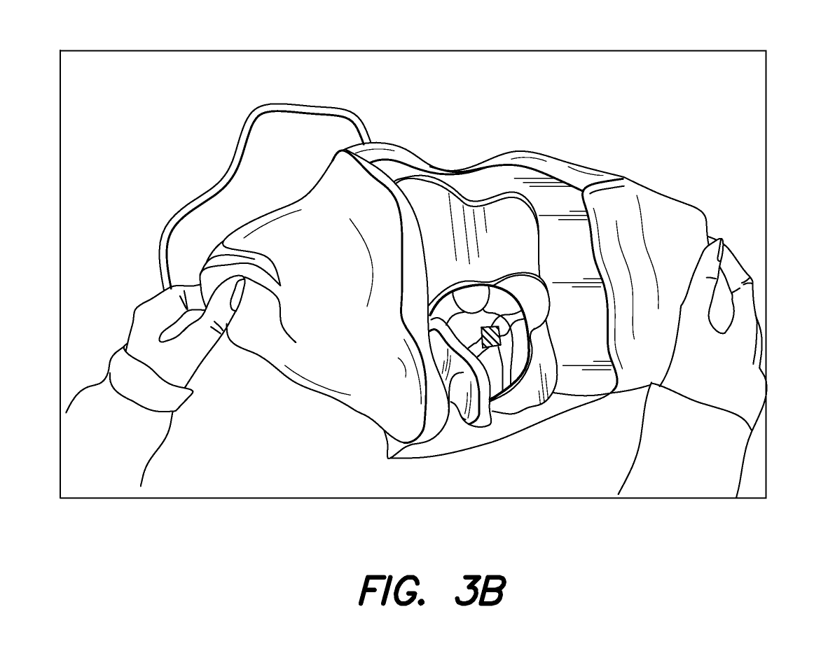

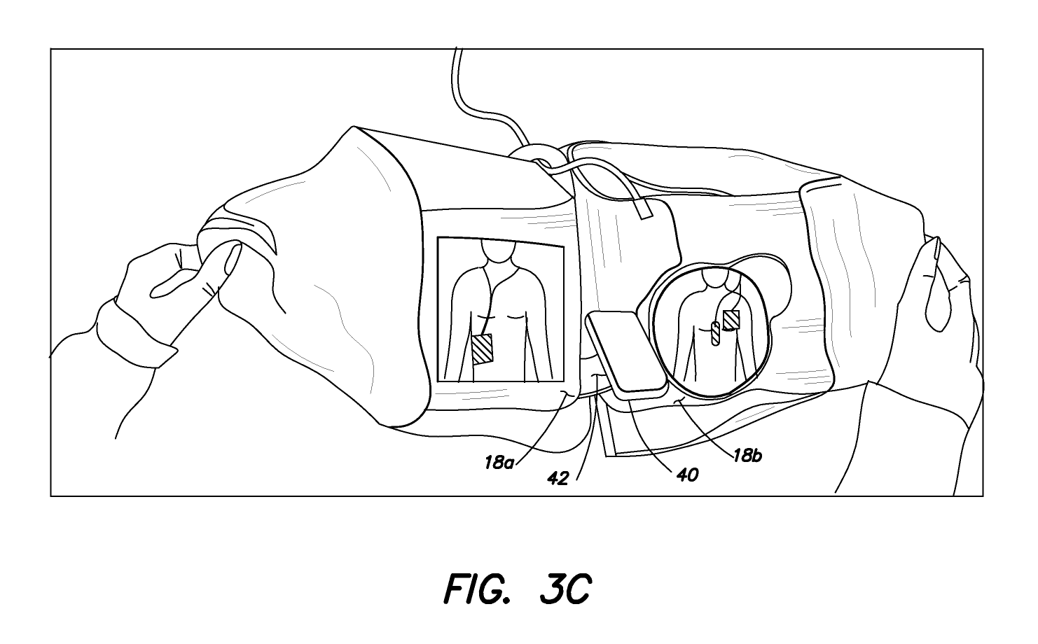

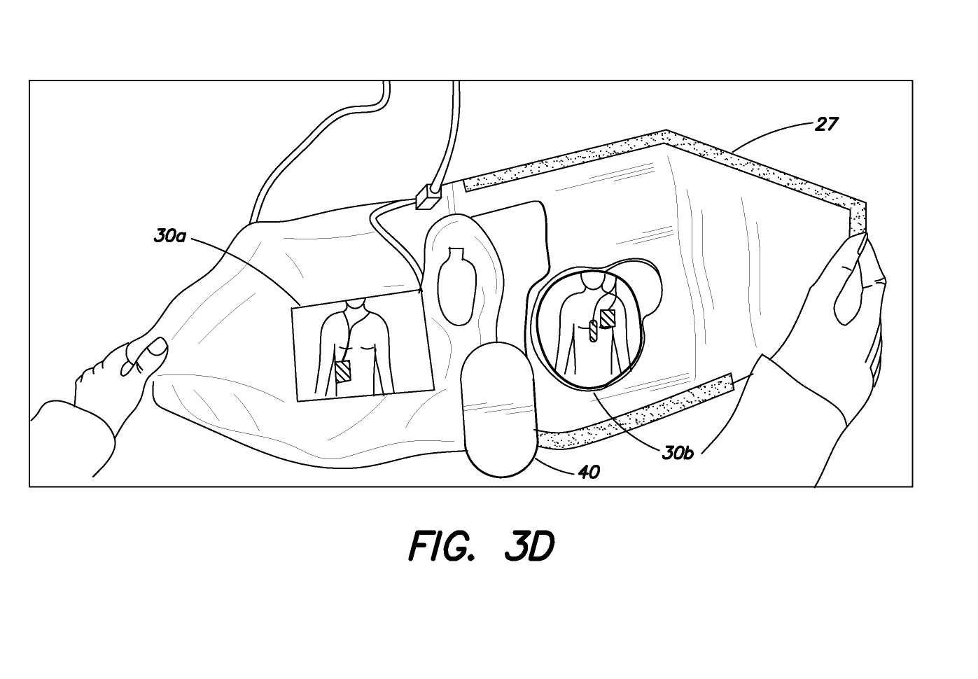

To open the electrode package 12, a user may grasp end portions 25 of the liner 18 and pull them apart. As the package 12 is opened, as illustrated in FIGS. 3A-3D, electrodes 30a and 30b and a CPR assistance device 40 become visible. As the liner 18 is opened further, the CPR assistance device 40 may move from a position internal to the pocket defined by the liner (FIG. 3B) to a position where a portion of the CPR assistance device 40 extends outside of a perimeter defined by the open liner 18 (FIG. 3D). The movement of the CPR assistance 40 device from the position inside the pocket defined by the liner to the position extending at least partially outside of the perimeter of the open liner 18 may be facilitated by a piece of material 42 coupled to the CPR assistance device 40 and having opposite ends coupled to opposite internal sides 18a, 18b of the liner 18 (FIG. 3C). The piece of material 42 extends from a folded orientation to an open orientation as the liner 18 is opened, pulling the CPR assistance device outward from a position within the pocket defined by the closed liner 18. The CPR assistance device 40 may be removably coupled to the piece of material 42 or to a coating on the piece of material 42 with an adhesive. The same adhesive used to removably couple the CPR assistance device 40 to the piece of material 42 may also be utilized to adhesively couple the CPR assistance device 40 to the body of a subject.

In some embodiments, the internal surface of the liner proximate the end portions 25 of the liner and/or a border of the internal surface of the liner may be provided with a sealing mechanism 27 which facilitates retaining the liner 18 in a folded configuration until pulled apart by a user. The sealing mechanism 27 may include, for example, one or more snaps, hook and loop fasteners, a low-tack uncured adhesive similar to that utilized in POST-IT® notes from 3M, one or more zipper connectors similar to those used in ZIPLOC® bags from S. C. Johnson & Son, or one or more other sealing mechanisms known in the art. The sealing mechanism may form an air-tight seal for the pocket defined by the closed liner which may facilitate the reduction or prevention of evaporation of any liquid from, for example, water-containing and/or conductive gel-containing adhesive layers of the electrodes 30a and 30b and/or CPR assistance device 40. In other embodiments, the liner 18 may be retained in a folded configuration by electrostatic attraction of internal surfaces of the liner 18 to one another.

The electrodes 30a and 30b and the CPR assistance device 40 may be positioned in the opened liner 18 in locations approximating the relative positions at which the electrodes 30a and 30b and the CPR assistance device 40 should be placed on a subject for treatment of the subject. For example, the electrode 30a, located on the left side of the opened liner 18 may be an electrode intended for placement on a lower right hand side of a subject's chest (a lower left side as viewed from the caregiver who applies the electrode). The electrode 30b, located on the right side of the opened liner 18 may be an electrode intended for placement on an upper left portion of a subject's chest (an upper right side as viewed from the caregiver who applies the electrode). The CPR assistance device 40, located between the electrodes 30a, 30b in the opened liner 18 may be intended for placement on the sternum of a subject between the electrodes 30a and 30b. The placement of the electrodes 30a and 30b and the CPR assistance device 40 within the opened liner 18 may provide a cue to a caregiver of where to position the electrodes 30a and 30b and the CPR assistance device 40 on a subject. The placement of the electrodes 30a and 30b and the CPR assistance device 40 within the opened liner 18 may thus reduce the amount of time that it takes the caregiver to decide where to place these components on the subject and reduce the time required to begin treatment of the subject

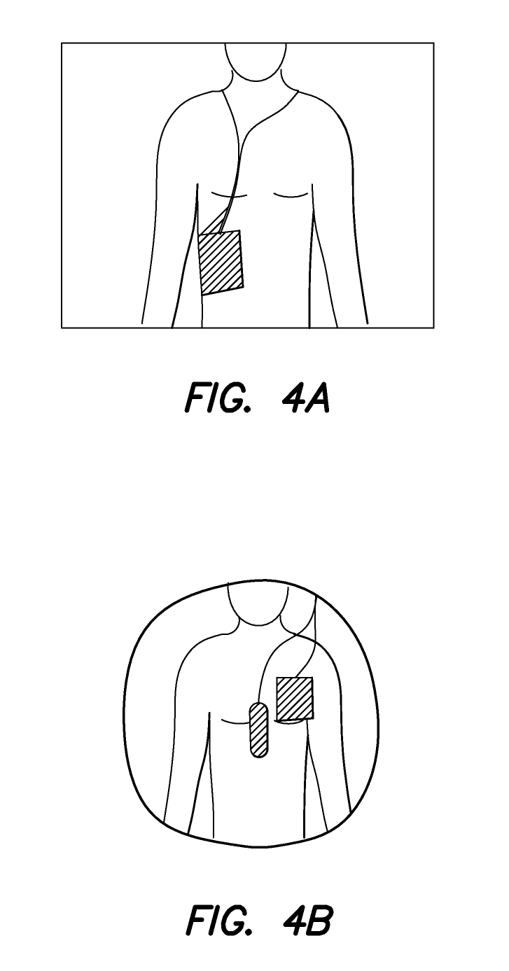

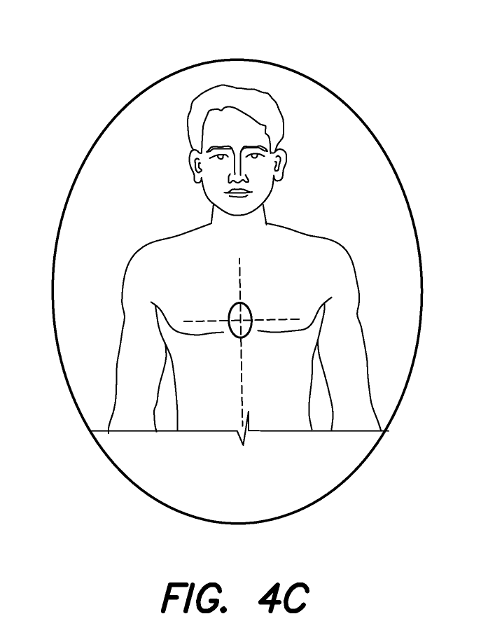

The electrodes 30a and 30b and the CPR assistance device 40 may be provided with labels, examples of which are illustrated in FIGS. 4A-4C, respectively, which provide a caregiver with further visual information regarding the proper placement of the electrodes 30a and 30b and the CPR assistance device 40 on a subject. These labels may further reduce the amount of time that it takes the caregiver to decide where to place these components on the subject and further reduce the time required to begin treatment of the subject. The electrodes 30a and 30b may be differently shaped, for example, one of the electrodes 30a, 30b may be substantially rectangular or substantially square shaped and the other of the electrodes 30a, 30b may be oval or circular. The shapes of the electrodes 30a, 30b may correspond to the shapes of the electrodes 30a, 30b shown on the labels of the respective electrodes 30a, 30b. The different shapes of the electrodes 30a, 30b may further help a caregiver distinguish one electrode from the other and further reduce the amount of time that it takes the caregiver to decide where to place the electrodes on the subject and further reduce the time required to begin treatment of the subject.

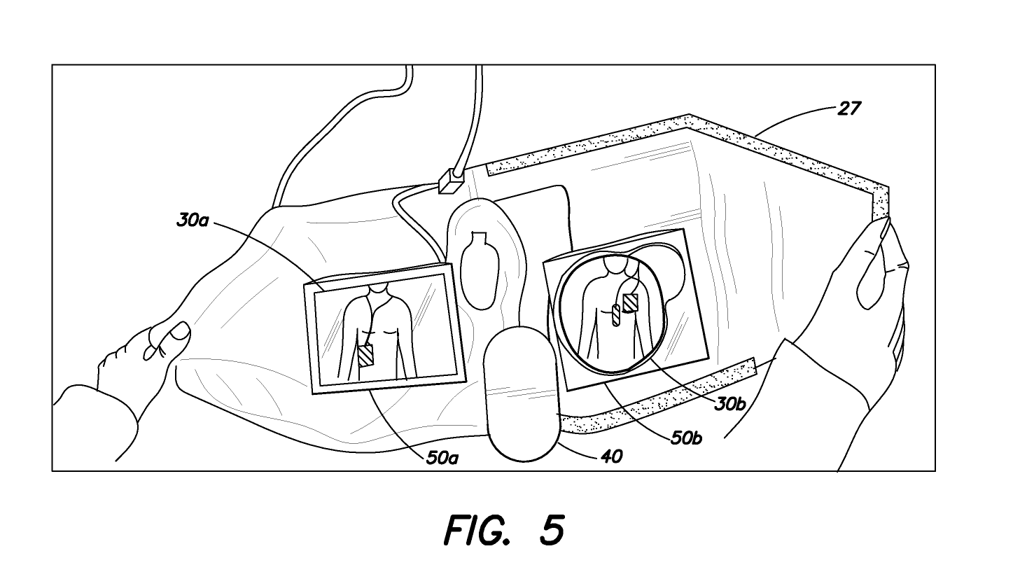

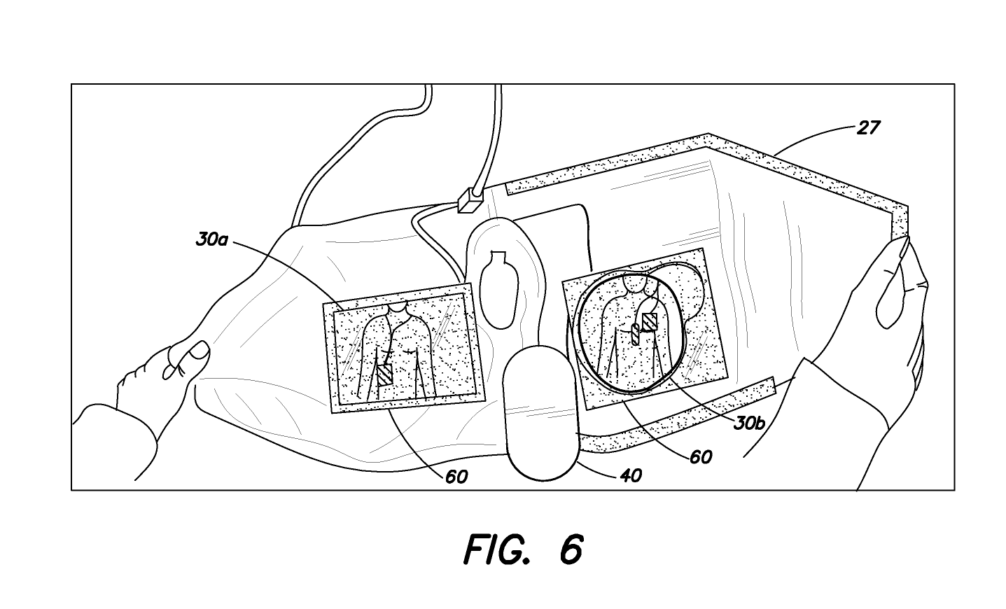

In some embodiments, the electrodes 30a and 30b may be disposed within sleeves 50a, 50b defined on inner sides of the liner 18 (FIG. 5). Alternatively or additionally, the inner sides of the liner 18 may be coated with a layer 60, for example, a polyester film (FIG. 6), to which adhesive bottom surfaces of the electrodes 30a, 30b may be adhered until removed by a caregiver.

The electrodes 30a, 30b may be single pad electrodes, each having a single electrical pad configured to deliver electrical energy to a subject. The electrodes 30a, 30b may, for example, be similar to those disclosed in co-pending U.S. patent application Ser. No. 13/079,336, published as Publication No. US20120253162, and incorporated herein by reference. Alternatively, one or both of the electrodes 30a, 30b may include multiple conductive pads to provide for monitoring, pacing, and/or defibrillation of a subject. One or both of the electrodes 30a, 30b may, for example, be similar to one of those disclosed in co-owned U.S. Pat. No. 8,185,199, incorporated herein by reference.

The CPR assistance device 40 may be, for example, a PocketCPR® CPR assistance device available from ZOLL Medical Corp. Examples of CPR assistance devices are disclosed in U.S. Pat. Nos. 7,245,974, 8,096,962, 6,390,996, 6,782,293, 7,108,665, 7,118,542, and 7,122,014. Aspects and embodiments disclosed herein are not limited to any particular forms of electrodes or CPR assistance devices unless explicitly set forth in the claims.

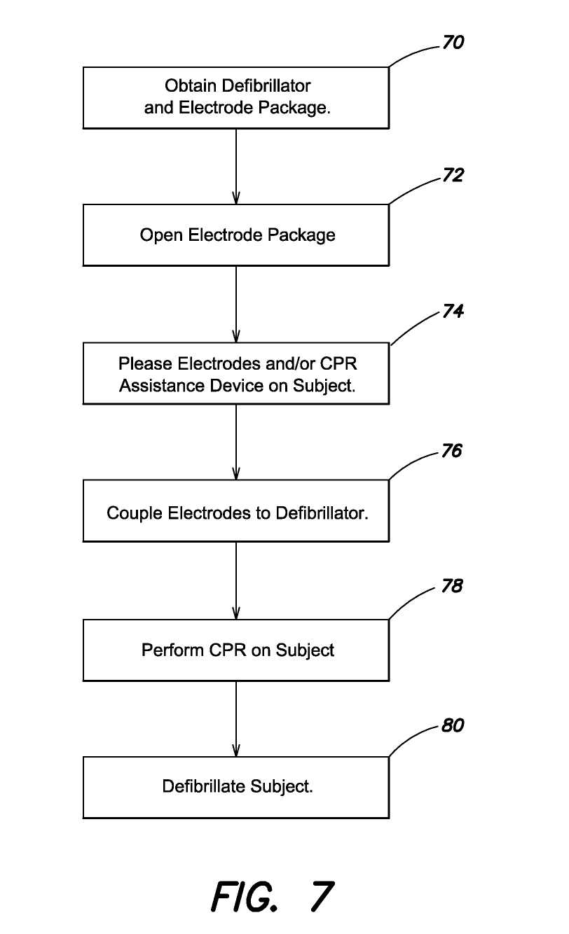

Another aspect includes a method of treating a subject with electrodes and/or a CPR assistance device included in a package as disclosed herein. A flowchart illustrating an embodiment of the method is shown in FIG. 7. In a first act of the method (act 70) upon identifying a subject experiencing cardiac distress, a first responder may summon emergency assistance and proceed to obtain a defibrillator, for example, an AED, and an electrode package. In some embodiments, the electrode package may be included with the defibrillator, for example, in a pocket or slot in the defibrillator. In act 72, the first responder opens the electrode package. When the electrode package is opened, electrodes and a CPR assistance device are positioned in the opened package in relative positions similar to those where the electrodes and CPR assistance device should be placed on the subject. The placement of the electrodes and CPR assistance device in the opened package thus facilitates the caregiver to quickly ascertain the correct placement of the electrodes and CPR assistance device on the subject. In act 74, the first responder places the electrodes and/or CPR assistance device on the subject. Diagrams on labels attached to the electrodes and CPR assistance device may further assist the first responder in selecting the proper locations for positioning the electrodes and CPR assistance device on the subject. In act 76, if the electrodes are not already coupled to the defibrillator, the first responder may electrically couple the electrodes and defibrillator, for example, with a quick connector coupled to a cable in electrical communication with the electrodes and, in some instances, also with the CPR assistance device. In some embodiments act 76 may be performed before act 74. The first responder then performs CPR as needed on the subject (act 78). The performance of CPR, for example, the frequency and depth of chest compressions may be guided by feedback from the CPR assistance device. If needed, the first responder may also defibrillate the subject (act 80). After defibrillation, the performance of CPR may be resumed. The determination of the need for defibrillation and the timing of the defibrillation may in some instances be determined by the defibrillator.

Having thus described several aspects of at least one embodiment of this invention, it is to be appreciated various alterations, modifications, and improvements will readily occur to those skilled in the art. For example, it is to be appreciated that any of the features of any of the embodiments disclosed herein may be combined or substituted for features of any other embodiment disclosed herein. Acts of the method disclosed may be performed in alternate orders and one or more acts may be added to or omitted from the method or substituted by one or more alternative acts. Such alterations, modifications, and improvements are intended to be part of this disclosure, and are intended to be within the scope of the invention. Accordingly, the foregoing description and drawings are by way of example only.

What is claimed is:

1. A therapy electrode package comprising:
a first therapy electrode disposed within the package;
a second therapy electrode disposed within the package; and
a CPR assistance device disposed within the package;
the first therapy electrode, the second therapy electrode, and the CPR assistance device arranged within the therapy electrode package such that when the electrode package is in an open configuration, the relative positions of the first to therapy electrode, the second therapy electrode, and the CPR assistance device approximate the relative positions at which the first therapy electrode, the second therapy electrode, and the CPR assistance device should be placed on a subject for treatment of the subject.

2. The therapy electrode package of claim 1, further comprising a liner which defines a pocket in which the first therapy electrode, the second therapy electrode, and the CPR assistance device are retained when the package is in a closed configuration.

3. The therapy electrode package of claim 2, wherein the liner includes a mechanism for maintaining the liner in a folded configuration absent application of a force to open the package.

4. The therapy electrode package of claim 3, wherein the mechanism provides the pocket with an air-tight seal.

5. The therapy electrode package of claim 2, wherein the CPR assistance device extends at least partially outside a perimeter defined by the liner while the package is in an open configuration.

6. The therapy electrode package of claim 5, wherein the liner includes a first side having a first internal surface and a second side including a second internal surface, and the CPR assistance device is adhered to a material having a first end coupled to the first internal surface and a second end coupled to the second internal surface.

7. The therapy electrode package of claim 6, wherein the material displaces the CPR assistance device from a position within the pocket to the position at least partially outside the perimeter defined by the liner upon opening the package.

8. The therapy electrode package of claim 2, wherein one of the first electrode and the second electrode are retained in a pocket defined within the package.

9. The therapy electrode package of claim 2, wherein one of the first electrode and the second electrode are removably adhered to a coating disposed on an inner surface of the liner.

10. The therapy electrode package of claim 1, disposed within a pocket defined in a defibrillator.

11. A method of providing treatment to a subject experiencing cardiac distress, the method comprising:
obtaining a defibrillator and an electrode package;
opening the electrode package to access electrodes and a CPR assistance device within the electrode package;
determining proper placement of the electrodes and CPR assistance device on the subject from relative positions of the electrodes and CPR assistance device within the opened package;
placing the electrodes and CPR assistance device on the subject; and
performing treatment on the subject, the treatment including at least one of application of CPR and delivery of a defibrillation pulse to the subject.

12. The method of claim 11, further comprising electrically coupling the electrodes to the defibrillator.

13. The method of claim 11, further comprising removing the electrode package from one of a pocket attached to the defibrillator and a slot defined in the defibrillator.

14. The method of claim 11, wherein opening the electrode package includes disconnecting portions of a sealing mechanism configured to maintain the therapy electrode package in a closed state until the therapy electrode package is opened by a user.

15. A therapy electrode package comprising:
a first therapy electrode;
a second therapy electrode; and
a CPR assistance device;
the therapy electrode package having a closed state in which the first therapy electrode, the second therapy electrode, and the CPR assistance device are disposed within the package, and having an open state, the CPR assistance device being moved by a portion of the package to a position extending at least partially beyond a periphery of the package upon a transition of the package from the closed state to the open state.

16. The therapy electrode package of claim 15, wherein the first therapy electrode, the second therapy electrode, and the CPR assistance device are arranged within the therapy electrode package such that when the electrode package is in the open state, the relative positions of the first therapy electrode, the second therapy electrode, and the CPR assistance device approximate the relative positions at which the first therapy electrode, the second therapy electrode, and the CPR assistance device should be placed on a subject for treatment of the subject.

17. The therapy electrode package of claim 16, wherein the portion of the package comprises a piece of material releasably coupled to the CPR assistance device and having opposite ends coupled to opposite internal sides of the package.

18. The therapy electrode package of claim 15, further including means for maintaining the therapy electrode package in a closed state until the therapy electrode package is opened by a user.

19. The therapy electrode package of claim 18, wherein the means for maintaining the therapy electrode package in the closed state forms an air-tight seal about a pocket defined in the package in the closed state, the pocket housing at least one of the first therapy electrode, the second therapy electrode, and the CPR assistance device.

20. The therapy electrode package of claim 15, wherein at least one of the first therapy electrode and the second therapy electrode are releasably adhesively retained on a coating of a liner of the therapy electrode package.
